(19) United States
(12) Patent Application Publication
(10) Pub. No.: US 2010/0197892 A1
(43) Pub. Date: Aug. 5, 2010

(54) ORGANOSILICON-FUNCTIONAL PHASE TRANSFER CATALYSTS

(76) Inventors: Kurt F. Brandstadt, Midland, MI (US); James H. Hand, Midland, MI (US); Thomas H. Lane, Midland, MI (US); Charles Leonard Liotta, Atlanta, GA (US); Charles Alan Eckert, Atlanta, GA (US); Andreas Sebastian Bommarius, Atlanta, GA (US); Reagan Charney, Atlanta, GA (US); Jason Patrick Hallett, London (GB); Karen Marie Polizzi, Exeter (GB); Pamela Pollet, Atlanta, GA (US)

Correspondence Address: DINSMORE & SHOHL LLP, FIFTH THIRD CENTER, ONE SOUTH MAIN STREET, SUITE 1300, DAYTON, OH, 45402-2023, US

(21) Appl. No.: 12/440,258

(22) PCT Filed: Sep. 12, 2007
(86) PCT No.: PCT/US07/78251

§ 371 Date: Mar. 6, 2009

(51) Int. Cl. C07K 1/00; C07F 7/10; C07K 14/00

(52) U.S. Cl. 530/345; 556/413; 530/402

(57) ABSTRACT

Organosilicon-functional phase transfer catalysts (PTCs) and methods for transferring immiscible molecules into a silicon-functional phase employing an organosilicon-functional PTC are provided.

The present invention relates to organosilicon-functional Phase Transfer Catalysts (PTCs) and methods for transferring immiscible molecules into a silicon-functional phase employing an organosilicon-functional Phase Transfer Catalyst (PTC).

For a reaction to take place efficiently it is desirable that all the reactants be present in the same phase. In practice, it can be quite challenging to bring reactants into a single solvent. For example, benzyl chloride and potassium cyanide can undergo nucleophilic substitution to form benzyl cyanide. Unfortunately, benzyl chloride and potassium cyanide are immiscible with each other and will not react efficiently unless they are present in the same phase. While this is an illustrative example, it is common in synthetic processes to have an organic substrate and an inorganic salt as reactants. Only a handful of polar solvents have the ability to dissolve both organic and inorganic substrates, among them dimethylsulfoxide (DMSO), dimethylformamide (DMF), and N-methylpyrolidone (NMP).

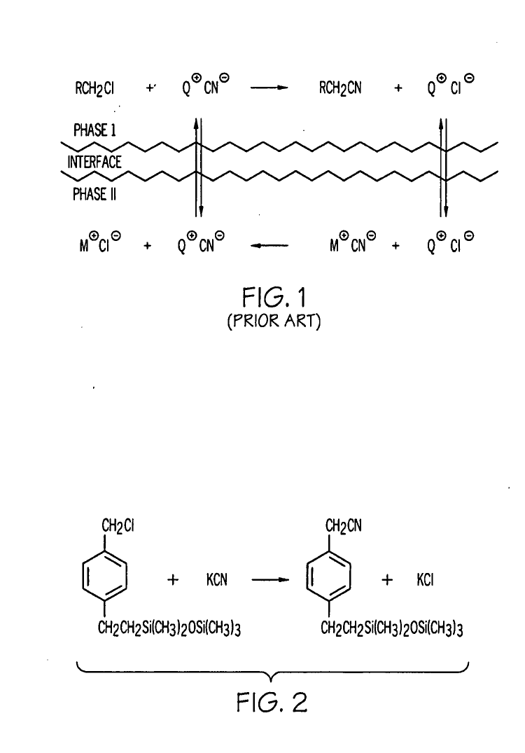

Unfortunately, dipolar aprotic solvents are such good solvents that the subsequent separation and isolation of the products can be very difficult. In addition, DMSO, DMF, or like dipolar aprotic solvents commonly have boiling points above 150° C., limiting the possibilities of distillation. Because the separation of the products is both difficult and costly, other synthetic routes have been devised to bypass the use of dipolar aprotic solvents. The most well-known example is phase transfer catalysis (PTC). A phase transfer catalyst is used to transfer inorganic salts and particularly anions from an aqueous or solid phase into an organic phase such as toluene or dichloromethane, in which the organic substrate is present, thereby allowing monophasic reaction while maintaining easy methods of separation (see FIG. 1).

Similar to inorganic/organic molecules, it can be quite challenging to bring molecules with very different polarities into a single phase. Currently, researchers have difficulty in reacting immiscible molecules such as hydrophobic organosilicon compounds and polar organic or inorganic reactants; or hydrophilic organosilicon compounds and non-polar organic molecules. Accordingly, there remains a need for a new type of phase transfer catalyst that could promote reactions between siloxane derivatives and reagents, which are immiscible with the siloxane derivative.

Embodiments of the present invention are directed to an organosilicon-functional Phase Transfer Catalyst (PTC) comprising the formula:

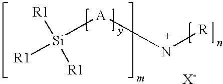

wherein each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0; A is a substituted or an unsubstituted hydrocarbon; R is a substituted or unsubstituted hydrocarbon; y is 0 or greater than 0; m is 1 to 4, n is 0 to 3, m+n=4; X⁻ is the anion associated with the quaternary amine cation; X⁻ comprises either a chloride, bromide, sulfate, or diacetamide anion. The immiscible substrates are transferred into a silicon-functional phase.

Embodiments of the present invention are also directed to an organosilicon-functional Phase Transfer Catalyst (PTC) methyl-tris-[3-(1,1,3,3,3-pentamethyl-disiloxanyl)-propyl]-ammonium X⁻ anion as depicted in FIG. 4A comprising the formula:

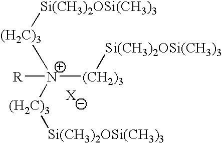

wherein R comprises a methyl or benzyl group, and X⁻ comprises chloride, bromide, sulfate or diacetamide.

Embodiments of the present invention are further directed to an organosilicon-functional Phase Transfer Catalyst (PTC) triethyl-{4-[2-(1,1,3,3,3-pentamethyl-disiloxanyl)-ethyl]-benzyl}-ammonium chloride as depict in FIG. 4B comprising the formula:

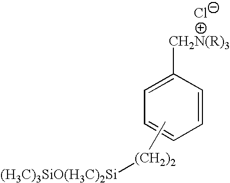

In other embodiments, the anion may comprises a bromide, sulfate, or diacetamide anion.

Embodiments of the present invention are further directed to an organosilicon-functional Phase Transfer Catalyst (PTC) comprising the formula:

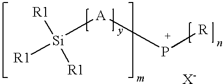

wherein: each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0; A is a substituted or an unsubstituted hydrocarbon substituent; R is a substituted or unsubstituted hydrocarbon substituent; y is 0 or greater than 0; m is 1 to 4, n is 0 to 3, m+n=4; X⁻ is the anion associated with the quaternary phosphonium cation; X⁻ comprises a chloride, bromide, sulfate, or diacetamide anion, or other commonly employed PTC anionic species.

Embodiments of the present invention are further directed to an organosilicon-functional Phase Transfer Catalyst (PTC) comprising a silicon functional crown ether.

Embodiments of the present invention are further directed to an organosilicon-functional Phase Transfer Catalyst (PTC) comprising a silicon functional polyethylene glycol.

Embodiments of the present invention are further directed to methods for transferring immiscible molecules into a silicon-functional phase comprising contacting an organosilicon-functional phase transfer catalyst (PTC) with a system comprising immiscible molecules, wherein the immiscible molecules are transferred into a silicon-functional phase.

In order that the embodiments of the invention may be more readily understood, reference will now be made, by way of example, to the accompanying drawings in which:

FIG. 1 is a schematic illustration of an example of a prior art phase transfer catalysis reaction;

FIG. 2 is an illustration of the reaction of 4-ethyl(1,1,3,3,3-pentamethyl-disiloxane) benzyl chloride with potassium cyanide;

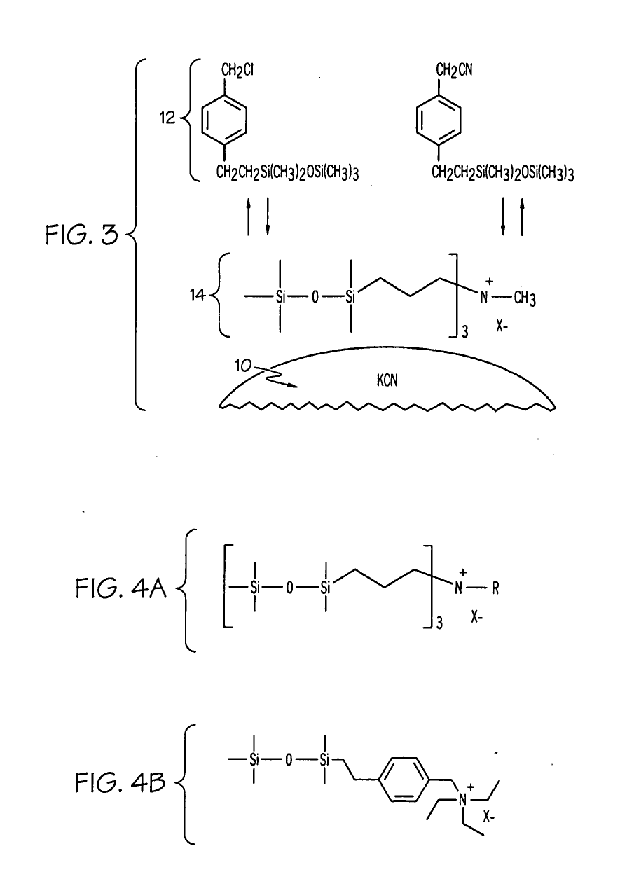

FIG. 3 is a schematic illustration of the role played by an organosilicon-functional phase transfer catalyst in a system that includes a solid phase that is immiscible with a siloxane phase.

FIGS. 4A and 4B illustrate chemical structures for additional embodiments of organosilicon-functional phase transfer catalysts of the present invention;

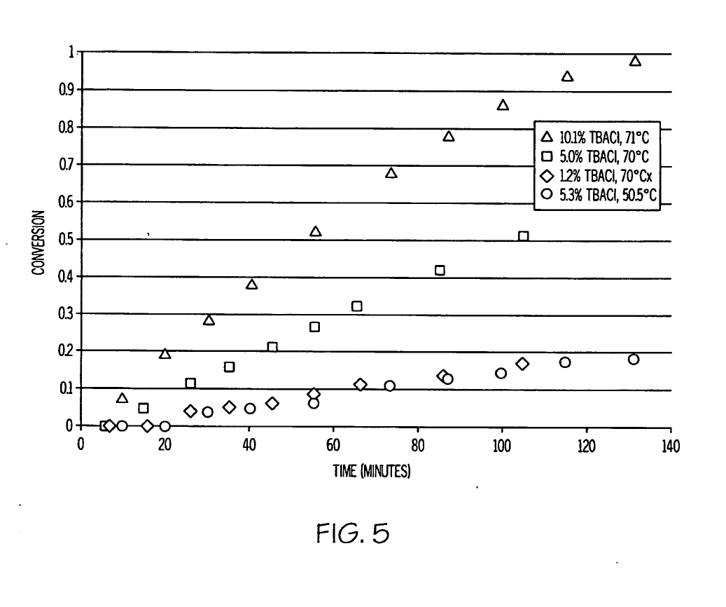

FIG. 5 is a graph of the time dependent behavior of the reaction of KOAc with siloxane electrophile and various concentrations of TBAC1 PTC;

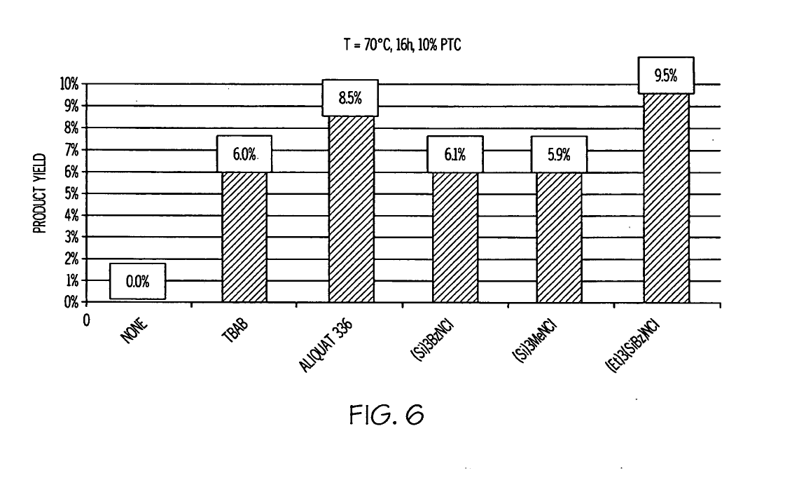

FIG. 6 is a graph comparing the respective product yields for five PTCs for the reaction of KCN with the siloxane electrophile under solventless conditions;

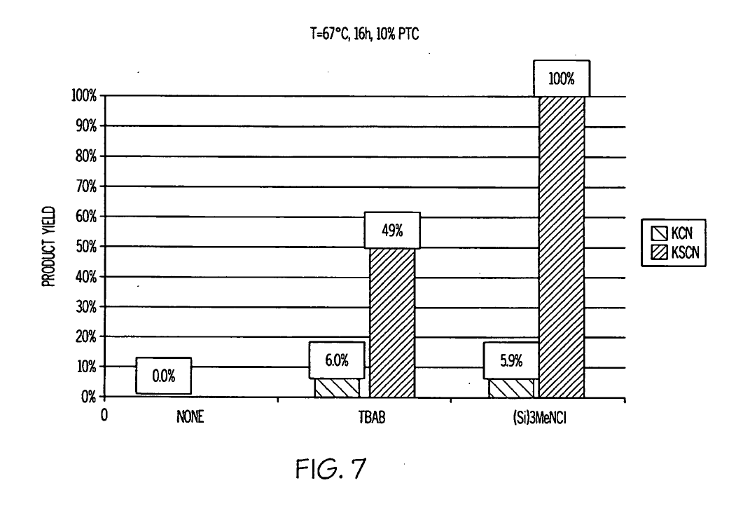

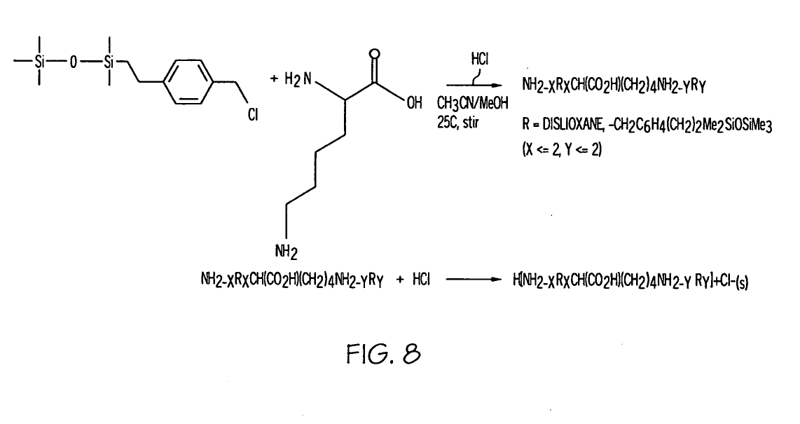

FIG. 7 is a graph comparing the respective product yield for two different nucleophiles (KCN and KSCN) with TBAB and (Et)₃ (SiBz) NCl; and

FIG. 8 is a schematic depiction for the coupling of p-SiBzCl with L-lysine.

Organosilicon-functional Phase Transfer Catalysts (PTCs) have been designed, which may be employed to transfer immiscible molecules into a silicon-functional phase to facilitate a monophasic reaction between the immiscible molecules. The organosilicon-functional PTCs broadly include, but are not limited to, (1) silicon-functional quaternary amines, (2) silicon-functional tetraalkyl phosphonium salts, (3) silicon-functional crown ethers (cryptates), and (4) silicon-functional polyethylene glycol.

Tetraalkyl phosphonium salts, which may contain aryl or siloxane architectures, are direct analogs of tetraalkyl ammonium salts, and work via the same principles of ion exchange and/or hydrogen bonding. In one embodiment, an organosilicon-functional Phase Transfer Catalyst (PTC) comprises the formula:

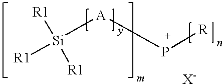

wherein: each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0; A is a substituted or an unsubstituted hydrocarbon substituent; R is a substituted or unsubstituted hydrocarbon substituent; y is 0 or greater than 0; m is 1 to 4, n is 0 to 3, m+n=4; X⁻ is the anion associated with the quaternary phosphonium cation; X⁻ comprises a chloride, bromide, sulfate, or diacetamide anion; or other commonly employed PTC anionic species. One skilled in the art will appreciate the various substituents that may substitute the hydrocarbon of A and/or R, any of which may be employed herein. Examples of substituents include, but are not limited to, ether-, alkoxy-, phenyl-, alkenyl, alkynyl-, unsaturated-functional hydrocarbon or a combination thereof;

Crown ethers, such as 18-crown-6, are a class of cyclic polyethers, usually derived from ethylene oxide or propylene oxide, thus containing 2-3 methylene units between each ether oxygen. The ether oxygens hydrogen bond to the cation, thus “dragging” an inorganic salt pair into the organic phase. In one embodiment, the organosilicon-functional Phase Transfer Catalyst (PTC) comprises a silicon functional crown ether. In one embodiment, the formula of the silicon functional crown ether comprises:

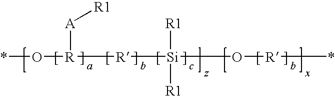

wherein R¹ and A are as defined for the ammonium compounds, R and R′ are substituted or unsubstituted hydrocarbon groups and a≧0, b≧0, c=0 or 1, z≧1, x≧0.

Polyethylene glycol comprises a non-cyclic analog of crown ether, which works by hydrogen bonding between the ether oxygens and the cationic center (cation or zwitterion), producing an ion pair. In one embodiment, an organosilicon-functional Phase Transfer Catalyst (PTC) comprises a silicon functional polyethylene glycol. In one embodiment, the formula of the silicon functional polyethylene glycol comprises:

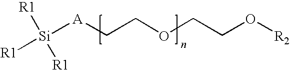

wherein each R¹ comprises linear, branched, or cyclic alkyl, unsaturated alkyl, aryl, hydroxy, alkoxy, hydrogen, or —(OSiR¹₂)ₚ—OSiR¹₃ where p is 0 or greater than 0; A is a substituted or an unsubstituted hydrocarbon, n≧0 and R² is hydrogen or a substituted or unsubstituted hydrocarbon. In another embodiment, the formula of the silicon functional polyethylene glycol comprises:

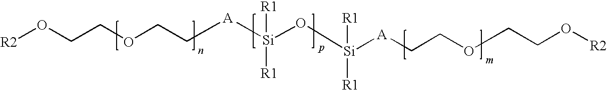

wherein each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0; A is a substituted or an unsubstituted hydrocarbon, n≧0, m≧0, p≧0, and R² is hydrogen or a substituted or unsubstituted hydrocarbon.

In one embodiment, an organosilicon-functional Phase Transfer Catalyst (PTC) comprises a silicon-functional quaternary amine. In one embodiment, the formula of the silicon-functional quaternary amine comprises the formula:

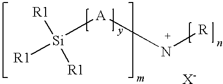

wherein: each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0; A is a substituted or an unsubstituted hydrocarbon substituent; R is a substituted or unsubstituted hydrocarbon substituent; y is 0 or greater than 0; m is 1 to 4, n is 0 to 3, m+n=4; X⁻ is the anion associated with the quaternary amine cation; X⁻ comprises a chloride, bromide, sulfate, or diacetamide anion, or other commonly employed PTC anionic species. One skilled in the art will appreciate the various substituents that may substitute the hydrocarbon of A and/or R, any of which may be employed herein. Examples of substituents include, but are not limited to, ether-, alkoxy-, phenyl-, alkenyl, alkynyl-, unsaturated-functional hydrocarbon or a combination thereof.

FIGS. 4A and B depict additional embodiments of organosilicon-functional phase transfer catalysts of the present invention and are, respectively, methyl-tris-[3-(1,1,3,3,3-pentamethyl-disiloxanyl)-propyl]-ammonium chloride ((Si)₃MeNCl) and triethyl-{4-[2-(1,1,3,3,3-pentamethyl-disiloxanyl)-ethyl]-benzyl}-ammonium chloride ((Si)₃BzNCl), wherein X⁻ comprises bromide, sulfate, diacetamide or other commonly employed PTC anionic species, which are known to one of ordinary skill in the art. The organosilicon-functional phase transfer catalyst in FIG. 4A is functionalized with three siloxane chains and substituted with an R group selected from the group consisting of an alkyl and an aryl. The organosilicon-functional phase transfer catalyst in FIG. 4B is functionalized with one aryl siloxane chain and substituted with three R groups selected from the group consisting of an alkyl and an aryl, producing a more organic-like catalyst as compared to the organosilicon-functional phase transfer catalyst of FIG. 4A.

FIGS. 4A and B are illustrative examples that are intended to demonstrate the diversity of the possible substitution patterns and the potential to adjust the properties by changing, for example, the length of the siloxane polymeric group, the number of siloxane groups, the number of organic groups, the functionality of the organic group, etc. By changing the substitution pattern, it is possible to adjust the hydrophobic/hydrophilic balance and, ultimately, to fine-tune the phase transfer catalyst to suit the immiscible molecules in a reaction.

The organosilicon-functional phase transfer catalysts as described herein may be used for transferring immiscible molecules into a silicon-functional phase. For example, FIG. 2 illustrates the reaction of 4-ethyl(1,1,3,3,3-pentamethyl-disiloxane)benzyl chloride with potassium cyanide. The reactants (the pentamethyldisiloxane-substituted benzyl chloride and the potassium cyanide) are very dissimilar and are immiscible molecules. In addition, it is almost impossible to dissolve these immiscible molecules into a single phase. The organosilicon-functional phase transfer catalysts as described herein enable these immiscible molecules to contact one another in a silicon-functional phase and react, overcoming their solubility dissimilarities.

FIG. 3 is a schematic representation of the role of an organosilicon-functional phase transfer catalyst as described herein in a system comprising a solid phase 10 which is immiscible with a siloxane phase 12. The organosilicon-functional phase transfer catalyst positions itself primarily at the surface of the ionic salt forming a silicon-functional phase 14. In this example, the silicon-functional phase 14 provides a welcoming environment for both reactants. If the system comprises two immiscible liquid phases, the organosilicon-functional phase transfer catalyst positions itself primarily at the interface of the two phases, which is analogous to the role described for solid/liquid phase interactions or within the silicon-functional phase as in classical liquid-liquid phase-transfer catalysis. One skilled in the art will appreciate that the immiscible molecules can be present in a variety of systems, any of which may be employed herein. Examples of systems, include, but are not limited to, neat-based systems and solvent-based systems. One skilled in the art will appreciate the various solvents that may be employed in the solvent-based systems. Examples of suitable solvents, include, but are not limited to, ethyl acetate, acetonitrile, toluene and chloroform.

One skilled in the art will also appreciate the various immiscible molecules that may be employed. Immiscible molecules comprise molecules with different polarities, organic molecules, inorganic molecules, or any immiscible combination thereof. In one embodiment, the immiscible molecules comprise a hydrophobic organosilicon compound and a polar organic or inorganic reactant. In another embodiment, the immiscible molecules comprise a hydrophilic organosilicon compound and a non-polar organic molecule. One skilled in the art will also appreciate the various inorganic molecules that may be employed. Inorganic molecules include, but are not limited to, KCN, KSCN and KOAc. One skilled in the art will also appreciate the various organic molecules that may be employed. Organic molecules included, but are not limited to, amino acids, polypeptides, proteins, and polyols.

One skilled in the art will also appreciate the various concentrations and/or amounts of immiscible molecules and/or PTC that may be employed in practicing embodiments of the present invention. In one embodiment, at least 1 mole % PTC relative to the limiting electrophile is used. In another embodiment, from about 1 to about 10 mole % PTC relative to limiting electrophile is used. One skilled in the art will also appreciate the various reaction temperatures that may be employed. In one embodiment, the temperature of the reaction is from about 25° C. to about 70° C.

As noted above, depending on the immiscible molecules and PTC employed, one skilled in the art will appreciate the various reaction conditions, reactant amounts and reaction steps that may be employed to bring the immiscible molecules together. In one embodiment, the reaction steps comprise: Add 0.0048 g TBACl to a 25 mL flask. Add 0.1204 g KCl to flask. Add 0.1688 g KOAc to flask. Add 3 mL EtOAc to flask. Add 0.1 mL decane to flask. Stir from about 200 to about 1100 rpm overnight at room temperature (overhead stirring, sealed against evaporation). Heat in oil bath to 70° C. and stir for 3 hours. Next add 0.1 mL of siloxane electrophile, seal and stir, with samples taken at predetermined intervals. 50 microliter samples are removed by pipet and diluted in 1.5 mL EtOAc and analyzed by GC-MS (calibrated using purchased standards).

Besides their phase transfer abilities, these compounds may find many applications as surface active agents, emulsions, and/or antimicrobial agents. An emulsion that involves an organosilicon-functional phase and an aqueous and/or organic phase can benefit from the organosilicon-functional phase transfer catalysts disclosed herein. For example, the head group could be ionic and the silicon-functional groups could create the “hydrophobic” domain. Again, the optimization of the properties of the product as phase transfer catalyst, surface active agent, and/or microbial agent can be done by simply modifying the substitution pattern as previously described.

### EXAMPLES

### Example 1

### Synthesis of Organosilicon-Functional Phase-Transfer Catalysts (PTCs)

This Example illustrates the synthesis and characterization of three novel siloxane-based phase-transfer catalysts for use in coupling immiscible hydrophilic and hydrophobic molecules, such as siloxanes with amino acids, polypeptides, or proteins.

Three different organosilicon-functional PTCs are synthesized: 1) tri(propylene pentamethyl disiloxy)methylammonium chloride; 2) tri(propylene pentamethyldisiloxy)benzylammonium chloride; and 3) triethyl(p-ethylene pentamethyldisiloxybenzyl)ammonium chloride ((Et)3(SiBz)NCl) (FIG. 4(B). Organosilicon-functional PTCs (1) and (2) are synthesized using pentamethyldisiloxane, triallyl amine and either methyl chloride or benzyl chloride. Organosilicon-functional PTC (3) is synthesized using triethyl amine and the siloxane electrophile, 2-pentamethyldisiloxy-p-ethyl benzyl chloride (p-SiBzCl). All structures are verified using NMR, ESI-MS and elemental analysis.

### Example 2

### Activity of Prior Art Phase-Transfer Catalysts Compared with Organosilicon-Functional Phase-Transfer Catalysts

This Example compares the three organosilicon-functional PTCs synthesized in Example 1 along with several organic prior art PTCs on a model nucleophilic displacement reaction involving a siloxane electrophile and various inorganic and organic ionic nucleophiles. This Example demonstrates that the organosilicon-functional PTC method is effective and that the organosilicon-functional PTCs generally outperform prior art organic PTCs under solventless conditions, while the organic PTCs are more effective when organic solvents are employed.

In order to simplify the analysis, the siloxane electrophile, 2-pentamethyldisiloxy-p-ethyl benzyl chloride (p-SiBzCl), is coupled with various inorganic and organic nucleophiles for analysis by GC-MS. The nucleophiles included potassium cyanide (KCN), potassium thiocyanate (KSCN), and potassium acetate (KOAc). The activity of this reaction is examined both neat and with 80% toluene or ethyl acetate as a solvent and also with various PTCs. Both commercially available organic-based PTCs tetrabutylammonium chloride (TBAC1), tetrabutylammonium bromide (TBAB), tetraoctylammonium chloride (TOACl) and trioctylmethylammonium chloride (Aliquat 336)) and the three novel siloxane-based PTCs are utilized. Pertinent control reactions are also performed.

Table 1 presents results for the reaction of p-SiBzCl with a variety of nucleophilic salts in ethyl acetate (EtOAc) as solvent. Pseudo-first order rate constants are calculated based on an excess of nucleophile. Results are analyzed by GC-FID with decane used as an internal standard. It is apparent from these data that tetrabutylammonium chloride (TBACl) is the most effective phase transfer agent, with the siloxane-based PTCs performing poorest. This result is somewhat expected because of the polar nature of the solvent system, which is less favorable for siloxane-based compounds. Additionally, the nature of the nucleophile plays a vital role both in the speed of conversion and also the magnitude of the PTC-related effects. The stronger nucleophile KOAc converts 100 times faster than KCN, and TBACl is nearly 70 times more effective than SiBz PTC for this nucleophile. Meanwhile, the rates vary by only a factor of 2.2 for the weaker nucleophile KSCN, which is a weaker nucleophile than KOAc and also less soluble in EtOAc. These results differ greatly from the solventless reactions discussed below.

TABLE 1
Pseudo-first order rate constants for the reaction of several nucleophiles
with p-siloxane electrophile and various PTCs at 70° C. and 900 rpm
stirring. 5x excess KOAc was used in all conditions and equimolar
KCl salt added to ensure constant ionic composition.

| | Pseudo-First Order Rate Constant (1/s) [k1] | | | |
|---|---|---|---|---|
| PTC (5%) | KCN | KSCN | KOAc | Lysine |
| None | No RxN | 8.0E−06 | No RxN | No RxN |
| TBACl | 2.1E−05 | 2.6E−05 | 5.7E−04 | 1.1E−05 |
| Aliquat 336 | 1.8E−06 | 2.6E−05 | 2.2E−04 | 1.6E−05 |
| (Si)3MeNCl | 1.2E−06 | 1.9E−05 | 2.0E−05 | 6.3E−06 |
| (Si)3BzNCl | 7.5E−06 | 1.2E−05 | 8.0E−06 | 4.3E−06 |

Table 2 presents results for the reaction of m-SiBzCl, a second isomer found in the starting material, in the same EtOAc reactions as in Table 1. Pseudo-first order rate constants followed similar trends for this isomer also, with rates generally slightly faster than for the para isomer.

TABLE 2
Pseudo-first order rate constants for the reaction of several nucleophiles
with m-siloxane electrophile and various PTCs at 70° C. and 900 rpm
stirring. 5x excess KOAc was used in all conditions and equimolar
KCl salt added to ensure constant ionic composition.

| | Pseudo-First Order Rate Constant (1/s) [k2] | | | |
|---|---|---|---|---|
| PTC (5%) | KCN | KSCN | KOAc | Lysine |
| None | No RxN | 7.7E−06 | No RxN | No RxN |
| TBACl | 2.8E−05 | 2.7E−05 | 6.5E−04 | 1.4E−05 |
| Aliquat 336 | 2.5E−06 | 2.6E−05 | 2.5E−04 | 1.7E−05 |
| (Si)3MeNCl | 2.5E−06 | 1.9E−05 | 2.3E−05 | 8.6E−06 |
| (Si)3BzNCl | 9.0E−06 | 1.3E−05 | 9.9E−06 | 5.7E−06 |

Table 3 presents sample results for the displacement of KOAc onto the siloxane electrophile catalyzed by TBAC1 under solventless conditions. 98% conversion at 70° C. is achieved in 105 min using 10% PTC (relative to electrophile) and 5× excess KOAc. The reaction is approximately 2.5-fold faster at 70° C. compared to 50° C. Activation energies of 95.1, 114.3, and 112.1 kJ/mol were determined for the EtOAc-based reactions with TBAC1, Aliquat 336, and (Si)₃MeNCl, respectively. The control reaction shows no conversion in 1100 minutes in the absence of PTC. FIG. 5 compares time-dependent behavior for each of these conditions.

TABLE 3
Reaction of KOAc with siloxane electrophile and various amounts of
TBACl PTC at 70 and 50° C. 5x excess KOAc is used in
all conditions.

| | | | Pseudo-first | Second-order |
|---|---|---|---|---|
| % PTC | Temperature | % Conversion | order rate | rate constant |
| (TBACl) | (° C.) | (105 min) | constant (s⁻¹) | (mL · s/mol) |
| 0 | 70 | 0 | 0 | 0 |
| 1.2 | 70 | 17 | 3.1 × 10⁻⁵ | 0.011 |
| 5.0 | 70 | 51 | 1.3 × 10⁻⁴ | 0.045 |
| 10.1 | 71 | 89 | 3.3 × 10⁻⁴ | 0.11 |
| 5.3 | 50.5 | 15 | 3.0 × 10⁻⁵ | 0.011 |

TABLE 4
Reaction of KOAc with siloxane electrophile and 5% PTC at 70, 50, and
30° C. 5x excess KOAc was used in all conditions.

| | Pseudo-First Order Rate | | |
|---|---|---|---|
| | Constant (1/s) [k1] | | |
| | KOAc | | |
| PTC (5%) | 30° C. | 50° C. | 70° C. |
| TBACl | 6.9E−06 | 6.6E−05 | 5.7E−04 |
| Aliquat 336 | 1.1E−06 | 1.4E−05 | 2.2E−04 |
| (Si)3MeNCl | — | 1.8E−05 | 2.0E−05 |

FIG. 6 compares five PTCs (two organics, three siloxanes) for the reaction of KCN with the siloxane electrophile under solventless conditions. At 70° C. and 16 h, the more nonpolar PTCs (Aliquat 336 and (Et)₃(SiBz)NCl, see FIG. 8) perform best, with 8.5 and 9.5% conversion.

FIG. 7 compares two different nucleophiles (KCN and KSCN) with TBAB and (Et)₃(SiBz)NCl. Conversions are much higher for KSCN, and the organosilicon-functional PTC outperforms TBAB, 100% to 49% conversion in 16 h at 67° C. Only the KSCN control shows any conversion (10% in 1100 min). Again, under solventless conditions, the less polar siloxane-based PTCs outperform the organic PTCs. This trend is the opposite of that found for EtOAc-based solvent systems. The polarity of the reaction medium clearly dictates which PTC will be most effective. The reaction rates are also 7-10 times higher for solventless systems due to the increase in substrate concentration. Reaction kinetics are more difficult to obtain accurately in the absence of solvent due to limited sample size, leading to more significant errors in the solventless rate measurements.

### Example 3

### Coupling of Siloxanes with an Amino Acid

This Example demonstrates the use of organosilicon-functional Phase Transfer Catalyst (PTC) for the purpose of amino acid-siloxane conjugation.

The model amino acid for coupling with the p-SiBzCl is L-lysine. The proposed reaction scheme is shown in FIG. 8. The reaction is performed in 1:1 (v:v) methanol:acetonitrile solvent mixture with equimolar reagents. The reaction produces 70% conversion in 22 hours, 82% after 42 hours, and 91% after 66 hours. By using no solvent and TBACl, 100% conversion is achieved in just 16 hours. This experiment used 10% PTC relative to p-SiBzCl and fivefold excess of L-lysine, similar to the PTC displacements. Product analysis is performed by NMR and ESI-MS and a distribution of alkylated products were observed.

Using 5% TBAC1 at 70° C., multiple runs in EtOAc yield a pseudo-first order rate constant of 1.06*10⁻⁵ s⁻¹ for this reaction, using 3× excess lysine. This rate is similar to that observed for the displacement of KOAc, the fastest nucleophile employed. This is an excellent result, as it indicates that lysine is a very active nucleophile for the coupling reaction. In the absence of any PTC, the reaction does not yield any conversion after 24 hrs.

Coupling of p-SiBzCl with six other amino acids (L-glutamic acid, D-phenylglycine, glycine, L-arginine, L-aspargine, L-glutamine) gave varying degrees of conversion in 16 hours. Coupling of p-SiBzCl with poly(L-lysine) showed 10% disappearance of siloxane by GC-FID in 16 hours and quantitative conversion after 72 hours.

The specific embodiments and examples set forth above are provided for illustrative purposes only and are not intended to limit the scope of the following claims. Additional embodiments of the invention and advantages provided thereby will be apparent to one of ordinary skill in the art and are within the scope of the claims.

1. An organosilicon-functional Phase Transfer Catalyst (PTC) comprising the formula: 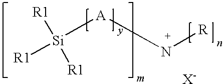
wherein:
each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0;
A is a substituted or an unsubstituted hydrocarbon;
R is a substituted or an unsubstituted hydrocarbon;
y is 0 or greater than 0;
m is 1 to 4, n is 0 to 3, m+n=4; and
X⁻ is the anion associated with the quaternary amine cation; and comprises chloride, bromide, sulfate, or diacetamide.

2. An organosilicon-functional Phase Transfer Catalyst (PTC) as claimed in claim 1 comprising methyl-tris-[3-(1,1,3,3,3-pentamethyl-disiloxanyl)-propyl]-ammonium X⁻ anion, and X⁻ comprises chloride, bromide, sulfate or diacetamide.

3. An organosilicon-functional Phase Transfer Catalyst (PTC) as claimed in claim 1 comprising triethyl-{4-[2-(1,1,3,3,3-pentamethyl-disiloxanyl)-ethyl]-benzyl}-ammonium chloride.

4. A method for transferring immiscible molecules into a silicon-functional phase comprising contacting an organosilicon-functional phase transfer catalyst (PTC) with a system comprising immiscible molecules, wherein the organosilicon-functional PTC comprises the formula: 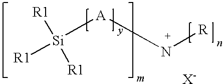
wherein:
each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0;
A is a substituted or an unsubstituted hydrocarbon;
R is a substituted or unsubstituted hydrocarbon;
y is 0 or greater than 0;
m is 1 to 4, n is 0 to 3, m+n=4; and
X⁻ is the anion associated with the quaternary amine cation; and X⁻ comprises chloride, bromide, sulfate, or diacetamide;
and wherein the immiscible substrates are transferred into a silicon-functional phase.

5. The method of claim 4, wherein the system is a neat or solvent-based system.

6. The method of claim 4, wherein the immiscible molecules comprise molecules with different polarities, organic molecules, inorganic molecules, or any immiscible combination thereof.

7. The method of claim 4, wherein the immiscible molecules comprise a hydrophobic organosilicon molecule and a polar organic or inorganic molecule.

8. The method of claim 4, wherein the immiscible molecules comprise a hydrophilic organosilicon molecule and a non-polar organic molecule.

9. The method of claim 6, wherein the immiscible molecules comprise organic molecules selected from the group consisting of amino acids, polypeptides, or proteins.

10. The method of claim 4, wherein the immiscible molecules react in the silicon-functional phase.

11. An emulsion comprising the organosilicon-functional PTC of claim 1.

12. An antimicrobial agent comprising the organosilicon-functional PTC of claim 1.

13. An organosilicon-functional Phase Transfer Catalyst (PTC) comprising the formula: 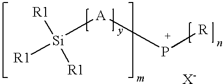
wherein: each R¹ comprises linear, branched, or cyclic alkyl, unsaturated alkyl, aryl, hydroxy, alkoxy, hydrogen, or —(OSiR¹₂)ₚ—OSiR¹₃, where p is 0 or greater than 0; A is a substituted or an unsubstituted hydrocarbon substituent; R is a substituted or unsubstituted hydrocarbon substituent; y is 0 or greater than 0; m is 1 to 4, n is 0 to 3, m+n=4; and X⁻ is the anion associated with the quaternary phosphonium cation; and X⁻ comprises chloride, bromide, sulfate, or diacetamide.

14. An organosilicon-functional Phase Transfer Catalyst (PTC) comprising a silicon functional crown ether having the formula:

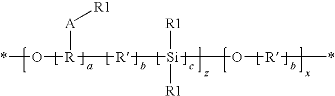

15. An organosilicon-functional Phase Transfer Catalyst (PTC) comprising a silicon functional polyethylene glycol having the formula: [chemical structure]
